(19) United States
(12) Patent Application Publication
(10) Pub. No.: US 2002/0192305 A1
(43) Pub. Date: Dec. 19, 2002

(54) Inflammatory mediation obtained from atractylodes lancea

(75) Inventors: David G. Corley, Chesterfield, MO (US); Mark Obukowicz, Kirkwood, MO (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Monsanto Company

(21) Appl. No.: 10/170,757

(22) Filed: Jun. 13, 2002

Related U.S. Application Data
(63) Continuation of application No. 09/272,363, filed on Mar. 19, 1999.

(51) Int. Cl. A61K 35/78

(52) U.S. Cl. 424/725; 424/773

(57) ABSTRACT

The present invention provides a method for inhibiting the activity of cyclooxygenase-2 and other proinflammatory factors in a mammal. The method comprises administering to the mammal, a therapeutically effective or prophylactically effective amount of an organic extract of Atractylodes lancea. The inhibitory effect of the organic extract of this invention on cyclooxygenase-2 activity is substantially greater than the inhibitory effect of the organic extract on cyclooxygenase-1 activity. The present invention also provides a method for treating a mammal having, or at risk for developing, a condition which is benefited by the inhibition of cyclooxygenase-2 or other proinflammatory factors. The method comprises administering to the mammal a therapeutically effective or prophylactically effective amount of the organic extract of Atractylodes lancea.

### FIELD OF THE INVENTION

This invention is in the field of nutritional and pharmaceutical agents for the management of inflammation related conditions. More specifically, this invention relates to the use of organic plant extracts to inhibit the activity of proinflammatory factors in inflamed tissue, as well as to prevent the incitation of proinflammatory factors in non-inflamed tissue.

### BACKGROUND OF THE INVENTION

Prostaglandins are known to play an important role in several conditions associated with the inflammation process. Consequently, significant efforts have been directed towards identification of agents which are capable of inhibiting prostaglandin synthesis.

Nonsteroidal antiinflammatory drugs (NSAIDS) are used to reduce pain and swelling associated with the inflammation process. These compounds function by inhibiting the synthesis of prostaglandins. However, because prostaglandins are also involved in maintaining proper gastrointestinal functioning, these compounds can have serious gastrointestinal side effects. Corticosteroids provide an alternative to NSAIDS; however, corticosteroids can result in even greater side effects than NSAIDs, especially when long term therapy is required.

NSAIDs are cyclooxygenase inhibitors. They interfere with the activity of cyclooxygenase enzymes and thereby inhibit the synthesis of prostaglandins. It is now known that there are two cyclooxygenase enzymes that are involved in prostaglandin synthesis. The cyclooxygenase enzyme responsible for prostaglandin synthesis in gastrointestinal tissue is called cyclooxygenase-1 (COX-1), while the cyclooxygenase enzyme responsible for prostaglandin synthesis in inflamed tissue is called cyclooxygenase-2 (COX-2).

It has been reported that NSAIDs may selectively inhibit either COX-1 or COX-2 activity. O'Neill et al., Molec. Pharmacol., 45, 245-254 (1994). This suggests that it should be possible to inhibit the activity of COX-2 without significantly inhibiting COX-1 activity. Thus, it should be possible to manage patients suffering from inflammation related conditions without causing significant gastrointestinal side effects.

Organic extracts of Atractylodes lancea have been shown to inhibit cyclooxygenase-1 activity in an enzymatic assay. Resch, et al., J. Nat. Prod., 61, 347-350 (1998). Methanol, dichloromethane and n-hexane extracts from rhizomes of Atractylodes lancea all showed inhibitory effects on COX-1 activity. The n-hexane extract was reported to contain an atractylochromene with significant COX-1 inhibitory effects. The reference does not disclose or suggest the use of organic extracts of Atractylodes lancea as selective COX-2 inhibitors, or administration of any purified and isolated compounds to mammals such as humans.

It would be very desirable to provide an agent that selectively inhibits the activity of COX-2 and other proinflammatory factors without significantly affecting the activity of COX-1.

### SUMMARY OF THE INVENTION

The present invention provides a method for inhibiting the activity of cyclooxygenase-2 and other proinflammatory factors in a mammal. The method comprises administering to the mammal a therapeutically-effective or prophylactically-effective amount of an organic extract of Atractylodes lancea. The inhibitory effect of the organic extract of this invention on the activity of cyclooxygenase-2 and other proinflammatory factors is substantially greater than the inhibitory effect of the organic extract on cyclooxygenase-1 activity.

The present invention also provides a method for managing a condition in a mammal which is benefited by the inhibition of cyclooxygenase-2 or other proinflammatory factors. The method comprises administering to the mammal a therapeutically-effective or prophylactically-effective amount of the organic extract of Atractylodes lancea.

### DETAILED DESCRIPTION OF THE INVENTION

As used herein, the term “purified” includes partially purified and completely purified. Thus, a “purified compound” may be either partially purified or completely purified. The term “proinflammatory factor” refers to substances (e.g., cytokines and eicosinoids) and processes (e.g., binding of inflammatory cells to protein matrixes) that are involved in the inflammation process. As used herein, the term “extract” includes crude extract, purified extract, and purified compounds obtained by purification of the extract.

It has been discovered that organic extracts of the rhizomes of the plant Atractylodes lancea exhibit selective inhibition of cyclooxygenase-2 (COX-2). The inhibitory effect is selective in that the inhibition of COX-2 is significantly greater than the inhibition of cyclooxygenase-1 (COX-1). These extracts also have been shown to inhibit other proinflammatory factors.

Consequently, organic extracts of Atractylodes lancea rhizomes may be used to selectively inhibit the activity of COX-2 and other proinflammatory factors in a mammal without causing an equivalent inhibition of COX-1 activity. Proinflammatory factors whose activity may be inhibited by the organic extracts of Atractylodes lancea include COX-2 activity, 15-lipoxygenase activity, thromboxane synthetase activity, inflammatory cell adhesion to fibronectin, inflammatory cell adhesion to VCAM-1, IL-1β cytokine release, IL-2 cytokine release, IL-6 cytokine release, Interferon-γ cytokine release, TNF-α cytokine release, TNF-α mediated PGE₂ release, IL-1a mediated PGE₂ release, NF-AT transcription of proinflammatory genes, and NF-κB transcription of proinflammatory genes.

The extracts of this invention may be used to manage a mammal having, or at risk for developing, a condition which is benefited by the inhibition of COX-2 or other proinflammatory factors. Conditions which may be benefited by the inhibition of COX-2 and other proinflammatory factors include, for example, general inflammation, arthritis, pain and cancer.

Preferably, the inhibitory effect of the extract of Atractylodes lancea on COX-2 is at least about two times greater than its inhibitory effect on COX-1. More preferably, the inhibitory effect on COX-2 is at least about 10 times greater than the inhibitory effect on COX-1.

Those of ordinary skill in the art of preparing pharmaceutical formulations can readily formulate pharmaceutical compositions having Atractylodes lancea extracts using known excipients (e.g., saline, glucose, starch, etc.). Similarly, those of ordinary skill in the art of preparing nutritional formulations can readily formulate nutritional compositions having Atractylodes lancea extracts. And those of ordinary skill in the art of preparing food or food ingredient formulations can readily formulate food compositions or food ingredient compositions having Atractylodes lancea extracts.

In addition, those of ordinary skill in the art can readily determine appropriate dosages that are necessary to achieve the desired therapeutic or prophylactic effect upon oral., parenteral, rectal and other administration forms. Typically, in-vivo models (i.e., laboratory mammals) are used to determine the appropriate plasma concentrations necessary to achieve a desired mitigation of inflammation related conditions.

The present invention provides a method for managing a condition in a mammal which is benefited by the inhibition of cyclooxygenase-2 or other proinflammatory factors. The method comprises administering to the mammal a therapeutically effective or prophylactically effective amount of the organic extract of Atractylodes lancea.

The organic extracts of the present invention may be obtained by extraction from Atractylodes lancea, in particular, from the rhizomes of Atractylodes lancea. In a desirable process, rhizomes of Atractylodes lancea are ground into a fine powder, the resultant powder is extracted with a solvent, and the extraction solvent is removed from the extract. If desired, the resultant extract may be further purified to yield a purified extract or one or more purified compounds.

The grinding step may be accomplished by any commonly known method for grinding a plant substance. For example, the rhizomes may be passed through a grinder to obtain a fine powder. After the rhizomes of Atractylodes lancea have been ground into a fine powder, they are combined with an extraction solvent.

The solution is stirred at a temperature, and for a period of time, that is effective to obtain an extract with the desired inhibitory effects on the activity of COX-2 and/or other proinflammatory factors. The solution should not be overheated, as this may result in degradation of the extract. The solution may be stirred at a temperature between about room temperature (25° C.) and the boiling point of the extraction solvent. Preferably, the solution is stirred at about room temperature.

The length of time during which the rhizome powder is exposed to the extraction solvent is not critical. Up to a point, the longer the rhizome powder is exposed to the extraction solvent, the greater is the amount of extract that may be recovered. Preferably, the solution is stirred for at least 1 minute, more preferably for at least 15 minutes, and most preferably for at least 60 minutes.

The extraction process of the present invention is desirably carried out using an organic solvent or a mixture of organic solvents. Organic solvents which may be used in the extraction process of the present invention include hydrocarbon solvents, ether solvents, chlorinated solvents, acetone, ethyl acetate, butanol, ethanol, methanol, isopropyl alcohol and mixtures thereof. Hydrocarbon solvents which may be used in the present invention include heptane, hexane and pentane. Ether solvents which may be used in the present invention include diethyl ether. Chlorinated solvents which may be used in the present invention include dichloromethane and chloroform. Preferably, the solvent is a nonpolar organic solvent, such as dichloromethane or hexane.

The relative amount of solvent used in the extraction process may vary considerably, depending upon the particular solvent employed. Typically, for each 100 grams of rhizome powder to be extracted, about 500 ml of extraction solvent would be used.

The organic solvent may be removed from the extract by any method well known in the field of chemistry for removing organic solvents from a desired product, including, for example, rotary evaporation.

It is believed that the inhibitors effect of the Atractylodes lancea extract of this invention on the activity of COX-2 and other proinflammatory factors is due to one or more compounds present in the extract. Compounds present in the extract which inhibit the activity of COX-2 and other proinflammatory factors may be isolated and purified by those of ordinary skill in the art using methods known in the art. For example, column chromatography and fractional distillation may be used to obtain pure compounds from the Atractylodes lancea extract of this invention.

The isolation and purification of particular compounds from organic extracts of Atractylodes lancea may be performed as described in Resch, et al., J. Nat. Prod., 61, 347-350 (1998), the entire contents of which are incorporated by reference herein. The methods disclosed therein may be used to isolate and purify compounds which exhibit selective inhibitory activity of COX-2 and other proinflammatory factors.

The examples which follow are intended to illustrate certain preferred embodiments of the invention, and no limitation of the invention is implied.

### PREPARATION EXAMPLE 1

Rhizomes of Atractylodes lancea (Oriental Ginseng and Gift, St. Louis, Mo.) were dried and sliced. The sliced rhizomes were ground into a fine powder using a coffee grinder. 100 grams of the resulting powder were added to 500 ml of dichloromethane and stirred at room temperature for 1 hour. The solvent was then removed by rotary evaporation, leaving 5.1 grams of a yellow-brown oil as the extract.

### EXAMPLE 1

### Inhibitory Effect of Organic Extract of Atractylodes lancea Rhizomes on COX-1 and COX-2 Activities

The organic extract obtained in Preparation Example 1 was evaluated for selective inhibition of COX-1 and COX-2. The COX-1 and COX-2 inhibition activities were determined in vitro by the art-recognized method described by Gierse et al., J. Biochem., 305, 479-484 (1995), summarized below.

Preparation of Recombinant COX Baculoviruses

Recombinant COX-1 was prepared by cloning a 2.0 kb fragment containing the coding region of human or murine COX-1 into a BamH1 site of the baculovirus transfer vector pVL1393 (Invitrogen) to generate the baculovirus transfer vectors for COX-1 in a manner similar to the method of D. R. O'Reilly et al., Baculovirus Expression Vectors: A Laboratory Manual (1992).

Recombinant baculoviruses were isolated by transfecting 4 μg of baculovirus transfer vector DNA into (2×10⁸) SF9 insect cells along with 200 mg of linearized baculovirus plasmid DNA by the calcium phosphate method. (See M. D. Summers and G. E. Smith, A Manual of Methods for Baculovirus Vectors and Insect Cell Culture Procedures, Texas Agric. Exp. Station Bull. 1555 (1987)). Recombinant viruses were purified by three rounds of plaque purification and high titer (10⁷-10⁸ pfu/ml) stocks of virus were prepared.

For large scale production, SF9 insect cells were infected in 10 liter fermentors (0.5×10⁶/ml) with the recombinant baculovirus stock such that the multiplicity of infection was 0.1. After 72 hours the cells were centrifuged and the cell pellet was homogenized in Tris/Sucrose (50 mM: 25%, pH 8.0) containing 1% 3-([(3-cholamidopropyl)dimethylammonio]-1-propanesulfonate (CHAPS). The homogenate was centrifuged at 10,000×G for 30 minutes, and the resultant supernatant was stored at −80° C.

Recombinant COX-2 was prepared by cloning a 2.0 kb fragment containing the coding region of human or murine COX-2 in the same manner as described above.

Assay for COX-1 and COX-2 Activities

COX-1 and COX-2 activities were assayed as PGE₂ formed/μg protein/time using ELISA to detect prostaglandin E₂ synthesized from arachidonic acid. CHAPS-solubilized insect cell membranes containing the COX-1 or COX-2 enzyme were incubated in a potassium phosphate buffer (50 mM, pH 8.0) containing epinephrine, phenol, and heme. Compounds were pre-incubated with the appropriate enzyme for 10-20 minutes. Arachidonic acid (10 μM) was then added to the mixture and the reaction was permitted to occur for ten minutes at room temperature (25° C.).

Any reaction between the arachidonic acid and the enzyme was stopped after ten minutes by transferring 40 μl of reaction mixture into 160 μl ELISA buffer and 25 μM indomethacin. The PGE₂ formed was measured by standard ELISA technology (Cayman Chemical).

A 200 mg sample of the extract obtained from Preparation Example 1 was dissolved in 2 ml of dimethyl sulfoxide (DMSO) for bioassay testing to determine the COX-1 and COX-2 inhibitory effects of the extract. The results of these bioassays are reported in Table 1.

TABLE 1

| Amount of | | |
|---|---|---|
| Extract | Percent Relative | Percent Relative |
| (mg/ml) | Control (COX-1) | Control (COX-2) |
| 0.50 | 33.3 | 4.9 |
| 0.17 | 86.3 | 10.9 |
| 0.056 | 155.2 | 17.8 |
| 0.019 | 152.2 | 25.4 |
| 0.0062 | 125.5 | 47.3 |
| 0.0021 | 104.7 | 63.8 |
| 0.00069 | 83.6 | 52.4 |
| 0.000023 | 77.2 | 79.8 |
| 0.000076 | 72.6 | 66.4 |
| 0.000025 | 66.0 | 61.4 |

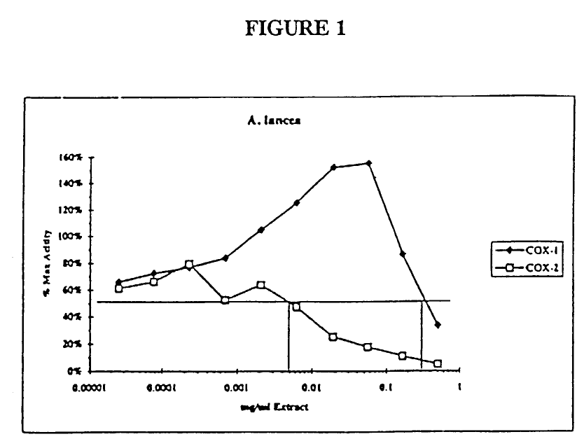

FIG. 1 is graph showing the data of Table 1. As can be seen in FIG. 1, the extract obtained in Example 1 has a much greater inhibitory effect on COX-2. than it does on COX-1. FIG. 1 shows that the COX-1 IC₅₀ of the extract was 350 μg/ml, whereas the COX-2 IC₅₀ of the extract was 5 μg/ml. Thus, the Atractylodes lancea extract of Example 1 had an inhibitory effect on COX-2 that was 70 times greater than its inhibitory effect on COX-1.

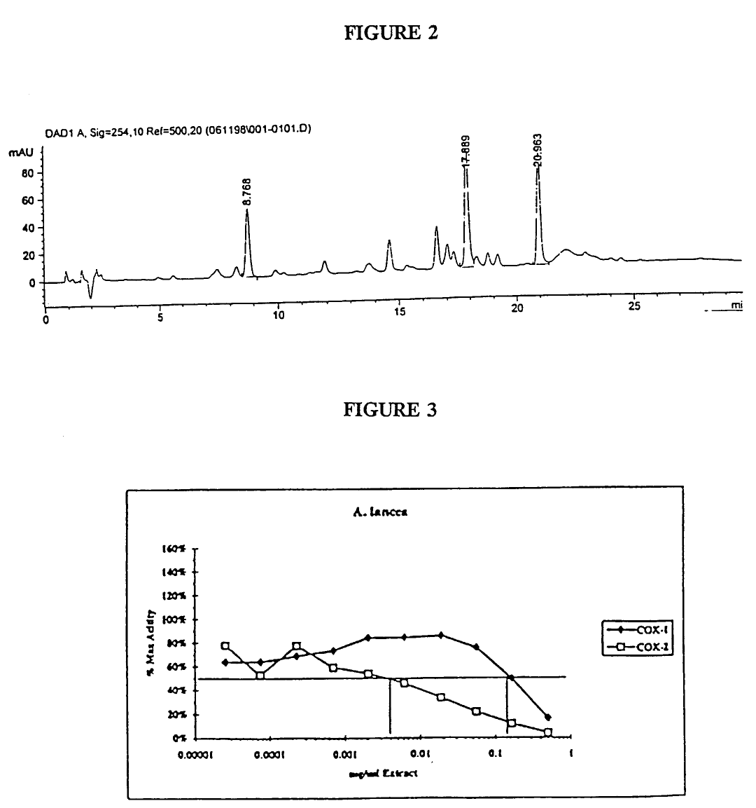

A sample of the organic extract of Preparation Example 1 in isopropanol (1 mg/ml) was prepared for HPLC analysis. The results of the HPLC analysis of Preparation Example 1 are shown in FIG. 2.

### PREPARATION EXAMPLE 2

Rhizomes of Atractylodes lancea (East Earth Herb, Eugene, Oreg.) were dried and sliced. The sliced rhizomes were ground into a fine powder using a coffee grinder. 100 grams of the resulting powder were added to 500 ml of dichloromethane and stirred at room temperature for 1 hour. The solvent was then removed by rotary evaporation, leaving 5.1 grams of a yellow-brown oil.

### EXAMPLE 2

A 200 mg sample of the extract obtained from Preparation Example 2 was dissolved in 2 ml of dimethyl sulfoxide (DMSO) and subjected to bioassay testing in the same manner as employed in Example 1. The results of these bioassays are reported in Table 2.

TABLE 2

| Amount of | | |
|---|---|---|
| Extract | Percent Relative | Percent Relative |
| (mg/ml) | Control (COX-1) | Control (COX-2) |
| 0.50 | 16.3 | 4.1 |
| 0.17 | 49.4 | 11.7 |
| 0.056 | 75.7 | 21.6 |
| 0.019 | 85.9 | 33.5 |
| 0.0062 | 84.7 | 45.5 |
| 0.0021 | 84.3 | 54.0 |
| 0.00069 | 73.1 | 59.1 |
| 0.00023 | 69.0 | 77.9 |
| 0.000076 | 64.3 | 52.9 |
| 0.000025 | 63.8 | 78.2 |

FIG. 3 is a graph showing the data of Table 2. As can be seen in FIG. 3, the extract obtained in Example 2 has a much greater inhibitory effect on COX-2 than it does on COX-1. FIG. 3 shows that the COX-1 IC₅₀ of the extract was 150 μg/ml, whereas the COX-2 IC₅₀ of the extract was 4 μg/ml. Thus, the Atractylodes lancea extract of Example 2 had an inhibitory effect on COX-2 that was 37 times greater than its inhibitory effect on COX-1.

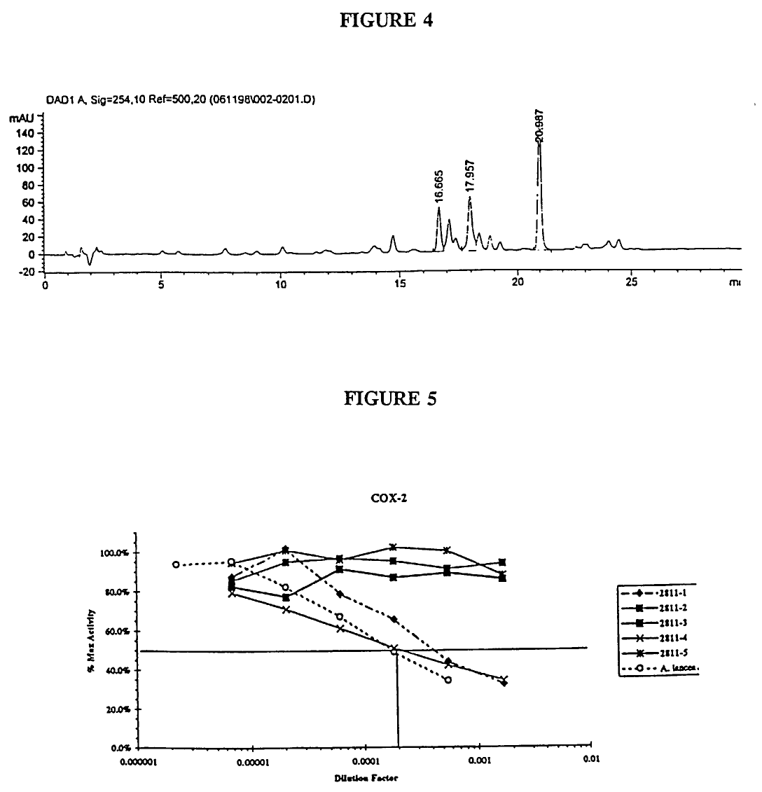

A sample of the organic extract of Preparation Example 2 in isopropanol (1 mg/ml) was prepared for HPLC analysis. The results of the HPLC analysis of Preparation Example 2 are shown in FIG. 4.

### EXAMPLE 3

A 200 mg sample of the Atractylodes lancea extract obtained from Preparation Example 2 was dissolved in 2 ml of DMSO. A-100 μl sample of the resultant solution was fractionated over a 500 mg C-18 Bond Elute SPE column as follows: the column was equilibrated with 100% methanol, followed by a 1:1 mixture of water to methanol. The 100 μl DMSO extract solution was added to 2.0 ml of a 1:1 water to methanol solution, and the resultant solution was added to the column. The column was then eluted with an additional 1.0 ml of a 1:1 mixture of water to methanol, thereby yielding a first fraction eluted with 3.0 ml of 1:1 water to methanol. The next fraction was eluted with 3.0 ml of a 1:4 water to methanol solution. The third fraction was eluted with 3.0 ml of pure methanol and the last fraction was eluted with 3.0 ml of dichloromethane. The four fractions were dried by rotary evaporation, and then the four fractions along with a crude Atractylodes lancea extract sample were dissolved in 100 μl DMSO for COX-2 bioassays. The results of these bioassays are reported in Table 3.

TABLE 3

| | COX-2 IC₅₀ |
|---|---|
| Elution Solvent | (100 μg/ml) |
| 1:1 H₂O:CH₃OH | >200 μg/ml |
| 1:4 H₂O:CH₃OH | >200 μg/ml |
| 100% CH₃OH | 18 μg/ml |
| 100% CH₂Cl₂ | >200 μg/ml |
| Crude extract | 16 μg/ml |

FIG. 5 is a graph showing the data of Table 3. As can be seen in FIG. 5, the pure methanol fraction displayed greater COX-2 inhibitory effects than any of the other purified fractions. The crude extract also displayed significant COX-2 inhibitory effects.

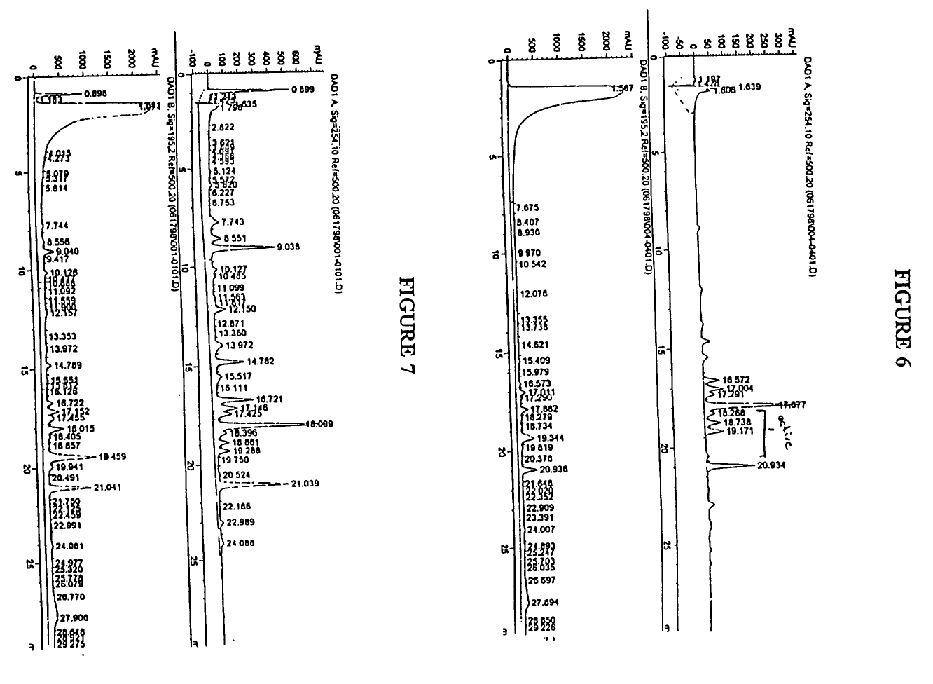

The pure methanol fraction and the positive control (i.e., crude extract) were analyzed using HPLC. The results of the HPLC analyses of the methanol fraction and the positive control are shown in FIGS. 6 and 7, respectively.

### EXAMPLE 4

A 5 g sample of the Atractylodes lancea extract of Preparation Example 1 was dissolved in 50 ml of dichloromethane and chromatographed over 100 g SiO₂ vacuum flash. After the 50 ml extract solution was applied to the column, the column was eluted with the following 200 ml fractions: 100% dichloromethane; 9:1 dichloromethane to methanol; 7:3 dichloromethane to methanol; 3:7 dichloromethane to methanol and 100% methanol. The five fractions were dried by rotary evaporation, and then the five fractions and a positive control (i.e., crude extract) were dissolved in DMSO (100 μg/ml) for COX-2 bioassays. The results of the bioassays are reported in Table 4.

TABLE 4

| | COX-2 IC₅₀ |
|---|---|
| Elution Solvent | (100 μg/ml) in DMSO |
| 100% CH₂Cl₂ | >500 μg/ml |
| 9:1 CH₂Cl₂:CH₃OH | 50 μg/ml |
| 7:3 CH₂Cl₂:CH₃OH | 6 μg/ml |
| 3:7 CH₂Cl₂:CH₃OH | 5 μg/ml |
| 100% CH₃OH | 50 μg/ml |
| Crude extract | 9 μg/ml |

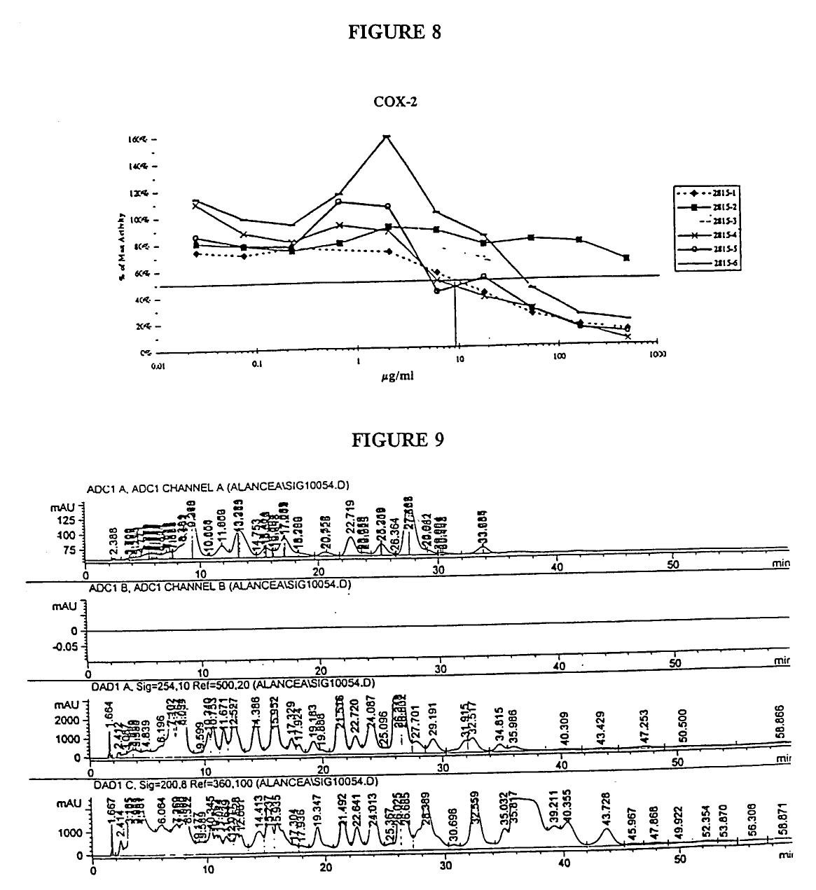

FIG. 8 is a graph showing the data of Table 4. As can be seen in FIG. 8, the third and fourth fractions (7:3 CH₂Cl₂ to CH₃OH and 3:7 CH₂Cl₂ to CH₃OH, respectively) displayed significant COX-2 inhibitory activity.

The 7:3 CH₂Cl₂ to CH₃OH fraction was analyzed by HPLC. The results of the HPLC analysis are shown in FIG. 9.

### EXAMPLE 5

### Inhibitory Effect Of Organic Extract of Atractylodes lancea Rhizomes on 15-Lipoxygenase Activity

The inhibitory effect of the organic extract obtained in Preparation Example 1 on 15-lipoxygenase was evaluated using methods described in Auerback et al., Anal. Biochem., 201, 375-380 (1992). A test solution of the extract was prepared at 100 μg/ml in 0.1% DMSO.

A 100 μg/ml sample of the test solution was incubated with 15 U of 15-lipoxygenase (obtained from rabbit reticulocytes) in a phosphate buffered saline solution at a pH of 7.4 and a temperature of 4° C. The reaction was initiated by addition of 256 μM linoleic acid as substrate and run for 10 minutes after which the reaction was terminated by addition of N-benzoyl leucomethylene blue (LMB). The level of 15-HETE was determined by measuring absorbance at 660 nm. The results of these assays are set forth in Table 5.

### EXAMPLE 6

### Inhibitory Effect Of Organic Extract of Atractylodes lancea Rhizomes on Thromboxane Synthetase Activity

The inhibitory effect of the organic extract obtained in Preparation Example 1 on Thromboxane Synthetase was evaluated using methods described in Fiddler et al., Circulation, 81 (Suppl) I69-I78 (1990). A test solution of the extract was prepared at 100 μg/ml in 0.1% DMSO.

A 100 μg/ml sample of the test solution was incubated with 1:200 dilution of thromboxane A₂ synthase (isolated from a microsomal fraction of rabbit platelets) and 5 ng prostaglandin G₂ as substrate in Tris buffer at a pH of 7.5 for 30 minutes at a temperature of 37° C. The thromboxane A₂ formed was immediately converted to thromboxane B₂ which was quantitated by a radioimmunoassay. The results of these assays are set forth in Table 5.

### EXAMPLE 7

### Inhibitory Effect Of Organic Extract of Atractylodes lancea Rhizomes on Fibronectin Mediated Cell Adhesion

The inhibitory effect of the organic extract obtained in Preparation Example 1 on Fibronectin mediated cell adhesion was evaluated using methods described in Nowlin, et al., J. Biol. Chem., 268, 20352-20359 (1993). Test solutions of the extract were prepared at 100 μg/ml, 10 μg/ml, 1 μg/ml, 0.1 μg/ml and 0.01 μg/ml in 0.1% DMSO.

These assays measured the adhesion of NRK 2 (normal rat kidney) cells to a fibronectin-coated well. Each of the test solutions in modified MEM-HEPES buffer at a pH of 7.4 was incubated for 30 minutes at a temperature of 37° C. The reactions were initiated by addition of NRK 2 cells (2×10⁶/ml) and incubated for 30 minutes. Each well was then washed 6 times with Dulbecco's PBS followed by addition of 5 μM calcein AM and a further 2 hour incubation period. Quantitation of fluorescent intensity resulting from interaction of calcein AM with cells attached to the fibronectin coated plate was read with a Cytofuor 2300 plate reader with B filter excitation at 485 nm and emission at 530 nm. The results of these assays are set forth in Table 5.

### EXAMPLE 8

### Inhibitory Effect Of Organic Extract of Atractylodes lancea Rhizomes on VCAM-1 Mediated Cell Adhesion

The inhibitory effect of the organic extract obtained in Preparation Example 1 on VCAM-1 mediated cell adhesion was evaluated using methods described in Stoltenborg, et al., J. Immunological Methods, 175, 59-68 (1994). Test solutions of the extract were prepared at 100 μg/ml, 10 μg/ml, 1 μg/ml, 0.1 μg/ml and 0.01 μg/ml in 0.1% DMSO.

These assays measured adherence of Jurkat (human T lymphoid) cells to wells coated with recombinant human VCAM-1 isolated from the membranes of infected SF9 cells. On the day of the assay, the plates were washed with Dubelco's PBS (DPBS) at a pH of 7.2. Each of the test solutions was incubated with Jurkat cells (0.5 to 1×10⁶/ml labeled with 5 μg/ml calcein AM) and 2.5 ng/ml. PMA (phorbol 12-myristate 13-acetate) in RPMI at a pH of 7.5 in the coated wells. The plates were incubated at 25° C. for 60 minutes without shaking and washed with DPBS. Adhesion was then quantitated by reading plates on a Cytoflour 2300. The results of these assays are set forth in Table 5.

### EXAMPLE 9

### Inhibitory Effect Of Organic Extract of Atractylodes lancea Rhizomes on IL-1β Cytokine Release

The inhibitory effect of the organic extract obtained in Preparation Example 1 on IL-1β cytokine release was evaluated using methods described in Welker, et al., International Arch of Allergy and Immunology, 109, 110-115 (1996). Test solutions of the extract were prepared at 100 μg/ml, 10 μg/ml, 1 μg/ml, 0.1 μg/ml and 0.01 μg/ml in 0.1% DMSO.

Each of the test solutions was incubated overnight with 25 ng/ml lipopolysaccharide (LPS) and stimulated human peripheral blood mononuclear leukocytes (PBMNL) in growth medium RPMI-1640 at a pH of 7.4 and a temperature of 37° C. The IL-1β cytokine production levels in the conditioned medium were quantitated using a sandwich ELISA kit. The results of these assays are set forth in Table 5.

### EXAMPLE 10

### Inhibitory Effect Of Organic Extract of Atractylodes lancea Rhizomes on IL-2 Cytokine Release

The inhibitory effect of the organic extract obtained in Preparation Example 1 on IL-2 cytokine release was evaluated using methods described in Welker, et al., International Arch of Allergy and Immunology, 109, 110-115 (1996). Test solutions of the extract were prepared at 100 μg/ml, 10 μg/ml, 1 μg/ml, 0.1 μg/ml and 0.01 μg/ml in 0.1% DMSO.

Each of the test solutions was incubated with 10 μg/ml Concanavalin-A (Con-A) and stimulated human peripheral blood mononuclear leukocytes (PBMNL) in growth medium RPMI-1640 at a pH of 7.4 and a temperature of 37° C. The IL-2 cytokine production levels in the conditioned medium were quantitated using a sandwich ELISA kit. The results of these assays are set forth in Table 5.

### EXAMPLE 11

### Inhibitory Effect Of Organic Extract of Atractylodes lancea Rhizomes on IL-2 Cytokine Release

The inhibitory effect of the organic extract obtained in Preparation Example 1 on IL-2 cytokine release also was evaluated using methods described in Koizumi, et al., 103, 469-475 (1986), which employ trypsinized Jurkat cells rather than PBMNL. Test solutions of the extract were prepared at 100 μg/ml, 10 μg/ml, 1 μg/ml, 0.1 μg/ml and 0.01 μg/ml in 0.1% DMSO.

Each of the test solutions was incubated overnight, in the presence or absence of co-stimulation by 1 μg/ml calcium ionophore (A23187) and 25 ng/ml PMA (phorbol 12-myristate 13-acetate), with trypsinized Jurkat (human T lymphoid) cells (2×10⁶/ml) suspended with 10% fetal bovine serum in RPMI-1640 at a pH of 7.4 and a temperature of 37° C. The cell suspension was subjected -to centrifugation, and the supernatant was evaluated for IL-2 release by use of an IL-2 immunoassay kit. The results of these assays are set forth in Table 5.

### EXAMPLE 12

### Inhibitory Effect Of Organic Extract of Atractylodes lancea Rhizomes on IL-6 Cytokine Release

The inhibitory effect of the organic extract obtained in Preparation Example 1 on IL-6 cytokine release was evaluated using methods described in Welker, et al., International Arch of Allergy and Immunology, 109, 110-115 (1996). Test solutions of the extract were prepared at 100 μg/ml, 10 μg/ml, 1 μg/ml, 0.1 μg/ml and 0.01 μg/ml in 0.1% DMSO.

Each of the test solutions was incubated overnight with LPS (25 ng/ml) and stimulated human peripheral blood mononuclear leukocytes (PBMNL) in growth medium RPMI-1640 at a pH of 7.4 and a temperature of 37° C. The IL-6 cytokine production levels in the conditioned medium were quantitated using a sandwich ELISA kit. The results of these assays are set forth in Table 5.

### EXAMPLE 13

### Inhibitory Effect Of Organic Extract of Atractylodes lancea Rhizomes on Interferon-γ Cytokine Release

The inhibitory effect of the organic extract obtained in Preparation Example 1 on Interferon-γ (IFN-γ) cytokine release was evaluated using methods described in the following references: (1) Cohen, et al., Am. J. Clin. Pathol. 105, 589-598 (1996); (2) Henderson, et al., TIPS, 13, 145-151 (1992); (3) Welker, et-al., International Arch of Allergy and Immunology, 109, 110-115 (1996); and (4) Elias, et al., J. Immunol., 138, 3812-3816 (1987). Test solutions of the extract were prepared at 100 μg/ml, 10 μg/ml, 1 μg/ml, 0.1 μg/ml and 0.01 μg/ml in 0.1% DMSO.

Each of the test solutions was incubated overnight with Concanavalin A (Con-A, 10 μg/ml)-stimulated human peripheral blood mononuclear cells (PBMNCS) in RPMI-1640 growth medium at a pH of 7.4 and a temperature of 37° C. IFN-γcytokine levels in the conditioned medium were then quantitated using a sandwich ELISA kit. The results of these assays are set forth in Table 5.

### EXAMPLE 14

### Inhibitory Effect Of Organic Extract of Atractylodes lancea Rhizomes on TNF-α Cytokine Release

The inhibitory effect of the organic extract obtained in Preparation Example 1 on TNF-α cytokine release was evaluated using methods described in the following references: (1) Cohen, et al., Am. J. Clin. Pathol. 105, 589-598 (1996); (2) Henderson, et al., TIPS, 13, 145-151 (1992); and (3) Welker, et al., International Arch of Allergy and Immunology, 109, 110-115 (1996). Test solutions of the extract were prepared at 100 μg/ml, 10 μg/ml, 1 μg/ml, 0.1 μg/ml and 0.01 μg/ml in 0.1% DMSO.

Each of the test solutions was incubated overnight with LPS (25 ng/ml) and stimulated human peripheral blood mononuclear cells (PBMNCs) in RPMI-1640 growth medium at a pH of 7.4 and a temperature of 37° C. TNF-α cytokine levels in the conditioned medium were then quantitated using a sandwich ELISA kit. The results of these assays are set forth in Table 5.

### EXAMPLE 15

### Inhibitory Effect Of Organic Extract of Atractylodes lancea Rhizomes on TNF-α Mediated PGE₂ Release

The inhibitory effect of the organic extract obtained in Preparation Example 1 on TNF-α mediated PGE₂ release was evaluated using methods described in Lenardo, et al., Cell, 58, 227-229 (1989). Test solutions of the extract were prepared at 100 μg/ml, 10 μg/ml, 1 μg/ml, 0.1 μg/ml and 0.01 μg/ml in 0.1% DMSO.

Each of the test solutions was incubated overnight in the presence or absence of 25 nM Tumor Necrosis Factor-α (TNF-α), with trypsinized HeLa (human epithelioid cervix carcinoma) S3 cells (2×10⁶/ml) suspended with 10% fetal bovine serum in MEM at a pH of 7.3 and a temperature of 37° C. The cell suspension of each well was then transferred to an Eppendorf vial, subjected to centrifugation, and the supernatant was evaluated for released PGE₂ (prostaglandin E2) by radioimmunoassay. The results of these assays are set forth in Table 5.

### EXAMPLE 16

### Inhibitory Effect Of Organic Extract of Atractylodes lancea Rhizomes on IL-1α Mediated PGE₂ Release

The inhibitory effect of the organic extract obtained in Preparation Example 1 on IL-1α mediated PGE2 release was evaluated using methods described in Maloff, et al., Clin. Chim. Acta, 180 73-78 (1989). Test solutions of the extract were prepared at 100 μg/ml, 10 μg/ml, 1 μg/ml, 0.1 μg/ml and 0.01 μg/ml in 0.1% DMSO.

Each of the test solutions was incubated overnight, in the presence or absence of 1 nM interleukin-1α (IL-1α), with trypsinized WI-38 (human diploid fibroblast lung) cells (10⁶/ml) suspended with 10% fetal bovine serum in MEM at a pH of 7.3 and a temperature of 37° C. The cell suspension of each well was then transferred to an Eppendorf vial, subjected to centrifugation, and the supernatant was evaluated for released PGE₂ (prostaglandin E₂) by radioimmunoassay. The results of these assays are set forth in Table 5.

### EXAMPLE 17

### Inhibitory Effect Of Organic Extract of Atractylodes lancea Rhizomes on NFKB

The inhibitory effect of the organic extract obtained in Preparation Example 1 on NFKB was evaluated using the methods described in Karttumen, et al., Proc. Nat'l. Acad. Sci. USA, 88, 3972-3976 (1991). Test solutions of the extract were prepared at 100 μg/ml, 10 μg/ml, 1 μg/ml, 0.1 μg/ml and 0.01 μg/ml in 0.1% DMSO.

Jurkat (human T lymphoid) cells transfected with a response element-lacZ reporter in which transcription of the β-galactosidase gene is directed by the binding site for the NF-κB transcription factor (κB-Z cells) were used in these assays. Each of the test solutions was incubated with κB-Z cells (2×10⁵) in the presence of 2 μM of a calcium ionophore (A23187) and 20 ng/ml PMA in RPMI buffer at a pH of 7.4 for 4 hours at 37° C. The cells were then centrifuged, resuspended in buffer and conversion of FDG (fluorescein di-β-D-galactopyranoside) to fluorescein by induced β-galactosidase activity was determined after overnight incubation in the dark at 25° C. Fluorescence intensity was measured using cytofluor (2300) plate reader with excitation at 485 nm and emission at 530 nm. The results of these assays are set forth in Table 5.

### EXAMPLE 18

### Inhibitory Effect Of Organic Extract of Atractylodes lancea Rhizomes on NF-AT

The inhibitory effect of the organic extract obtained in Preparation Example 1 on NF-AT was evaluated using the methods described in Emmel, et al., Science, 246, 1617-1620 (1989). Test solutions of the extract were prepared at 100 μg/ml, 10 μg/ml, 1 μg/ml, 0.1 μg/ml and 0.01 μg/ml in 0.1% DMSO.

Jurkat (human T lymphoid) cells transfected with a response element-lacZ reporter in which transcription of the β-galactosidase gene is directed by the binding site for the NFAT-1 transcription factor were used for the assays. Each of the test solutions was incubated with cells (2×10⁵) in the presence of 2 μM of a calcium ionophore (A23187) and 20 ng/ml PMA in RPMI buffer at a pH of 7.4 for 4 hours at 37° C. The cells were then centrifuged and resuspended in buffer, and conversion of FDG (fluorescein di-β-D-galactopyranoside) to fluorescein by induced β-galactosidase activity was determined after overnight incubation in the dark at 25° C. Fluorescence intensity was measured using a Cytofluor (2300) plate reader with excitation at 485 nm and emission at 530 nm. The results of these assays are set forth in Table 5.

TABLE 5

| | A. lancea | | |
|---|---|---|---|
| | extract conc. | % | IC₅₀ |
| Proinflammatory Factor | (μg/ml) | Inhibition | (μg/ml) |
| 15-Lipoxygenase | 100 | 50 | |
| Thromboxane Synthetase | 100 | 88 | |
| Cell Adhesion (Fibronectin) | 100 | 116 | 5.4 |
| Cell Adhesion (Fibronectin) | 10.0 | 65 | |
| Cell Adhesion (Fibronectin) | 1.0 | 13 | |
| Cell Adhesion (Fibronectin) | 0.1 | 15 | |
| Cell Adhesion (Fibronectin) | 0.01 | 10 | |
| Cell Adhesion (VCAM-1) | 100 | 109 | 9.9 |
| Cell Adhesion (VCAM-1) | 10.0 | 52 | |
| Cell Adhesion (VCAM-1) | 1.0 | −11 | |
| Cell Adhesion (VCAM-1) | 0.1 | 9 | |
| Cell Adhesion (VCAM-1) | 0.01 | 13 | |
| Cytokine Release (IL-1β) | 100 | 110 | 7.2 |
| Cytokine Release (IL-1β) | 10.0 | 85 | |
| Cytokine Release (IL-1β) | 1.0 | −8 | |
| Cytokine Release (IL-1β) | 0.1 | −3 | |
| Cytokine Release (IL-1β) | 0.01 | −9 | |
| Cytokine Release (IL-2) PBMNL | 100 | 102 | 0.4549 |
| Cytokine Release (IL-2) PBMNL | 10.0 | 89 | |
| Cytokine Release (IL-2) PBMNL | 1.0 | 45 | |
| Cytokine Release (IL-2) PBMNL | 0.1 | 37 | |
| Cytokine Release (IL-2) PBMNL | 0.01 | 20 | |
| Cytokine Release (IL-2) Jurkat | 100 | 87 | 70.6 |
| Cytokine Release (IL-2) Jurkat | 10.0 | −13 | |
| Cytokine Release (IL-2) Jurkat | 1.0 | −11 | |
| Cytokine Release (IL-2) Jurkat | 0.1 | −9 | |
| Cytokine Release (IL-2) Jurkat | 0.01 | −9 | |
| Cytokine Release (IL-6) | 100 | 100 | 2.7 |
| Cytokine Release (IL-6) | 10.0 | 99 | |
| Cytokine Release (IL-6) | 1.0 | 3 | |
| Cytokine Release (IL-6) | 0.1 | −4 | |
| Cytokine Release (IL-6) | 0.01 | −2 | |
| Cytokine Release (Interferon-γ) | 100 | 99 | 5.7 |
| Cytokine Release (Interferon-γ) | 10.0 | 68 | |
| Cytokine Release (Interferon-γ) | 1.0 | 9 | |
| Cytokine Release (Interferon-γ) | 0.1 | −2 | |
| Cytokine Release (Interferon-γ) | 0.01 | 4 | |
| Cytokine Release (TNF-α) | 100 | 107 | 9.3 |
| Cytokine Release (TNF-α) | 10.0 | 59 | |
| Cytokine Release (TNF-α) | 1.0 | −18 | |
| Cytokine Release (TNF-α) | 0.1 | −13 | |
| Cytokine Release (TNF-α) | 0.01 | −2 | |
| TNF-α Mediated PGE₂ Release | 100 | 90 | |
| TNF-α Mediated PGE₂ Release | 10.0 | 20 | |
| TNF-α Mediated PGE₂ Release | 1.0 | −5 | |
| TNF-α Mediated PGE₂ Release | 0.1 | −2 | |
| TNF-α Mediated PGE₂ Release | 0.01 | 0 | |
| IL-1α Mediated PGE₂ Release | 100 | 110 | |
| IL-1α Mediated PGE₂ Release | 10.0 | 44 | |
| IL-1α Mediated PGE₂ Release | 1.0 | 20 | |
| IL-1α Mediated PGE₂ Release | 0.1 | 8 | |
| IL-1α Mediated PGE₂ Release | 0.01 | −1 | |
| NFκB transcription | 100 | 96 | 7 |
| NFκB transcription | 10.0 | 59 | |
| NFκB transcription | 1.0 | 11 | |
| NFκB transcription | 0.1 | 7 | |
| NFκB transcription | 0.01 | −13 | |
| NF-AT transcription | 100 | 96 | 8.8 |
| NF-AT transcription | 10.0 | 54 | |
| NF-AT transcription | 1.0 | 7 | |
| NF-AT transcription | 0.1 | −12 | |
| NF-AT transcription | 0.01 | −10 | |

### EXAMPLE 19

A mammal having or at risk for developing a condition which is benefited by inhibition of the activity of COX-2 or other proinflammatory factors may be treated with a therapeutically or prophylactically effective amount of the Atractylodes lancea extract of this invention. The therapeutically effective or prophylactically effective amount of Atractylodes lancea extract may be administered to the mammal by any of the known routes for administering a pharmaceutical agent to a patient, including parenterally, orally and rectally. The Atractylodes lancea extract may be administered using a dosing regimen which is effective to inhibit the activity of COX-2 or other proinflammatory factors in the mammal for a desired period of time. The proper dosage amount may be determined by routine experimentation using methods known in the art.

Other variations and modifications of this invention will be obvious to those skilled in the art. This invention is not limited, except as set forth in the claims.

What is claimed is:

1. A method for inhibiting the activity of a proinflammatory factor in a mammal, said method comprising the step of administering to the mammal an organic extract of Atractylodes lancea.

2. A method according to claim 1, wherein said proinflammatory factor is selected from the group consisting of cyclooxygenase-2, 15-lipoxygenase, thromboxane synthetase, inflammatory cell adhesion to fibronectin, inflammatory cell adhesion to VCAM-1, IL-1β cytokine release, IL-2 cytokine release, IL-6 cytokine release, Interferon-γ cytokine release, TNF-α cytokine release, TNF-α mediated PGE₂ release, IL-1α mediated PGE₂ release, NF-AT transcription of proinflammatory genes, and NF-κB transcription of proinflammatory genes.

3. A method according to claim 1, wherein said proinflammatory factor is cyclooxygenase-2.

4. A method according to claim 3, wherein the inhibitory effect of said extract on cyclooxygenase-2 is greater than or equal to about 2 times greater than the inhibitory effect of said extract on cyclooxygenase-1.

5. A method according to claim 3, wherein the inhibitory effect of said extract on cyclooxygenase-2 is greater than or equal to about 10 times greater than the inhibitory effect of said extract on cyclooxygenase-1.

6. A method according to any of claims 1-5, wherein said organic extract is a purified compound obtained by a process comprising the steps of:
(i) exposing rhizomes of Atractylodes lancea to an organic solvent under conditions appropriate to remove a proinflammatory factor inhibitory extract from said rhizomes; and
(ii) isolating said proinflammatory factor inhibitory extract.

7. A method according to claim 6, wherein step (i) comprising mixing said rhizomes with said solvent and stirring the resultant mixture at a temperature between about 25° C. and the boiling point of said solvent for at least one minute.

8. A method according to claim 7, wherein said solvent is an organic solvent.

9. A method according to claim 8, wherein said organic solvent is selected from the group consisting of hydrocarbon solvents, ethers, chlorinated solvents, acetone, ethyl acetate, butanol, ethanol, methanol, isopropyl alcohol and mixtures thereof.

10. A method according to claim 9, wherein said nonpolar organic solvent is dichloromethane.

11. A method according to claim 9, wherein step (ii) comprises separating said solvent from said organic extract by evaporating said solvent.

12. A method according to claim 6, comprising administering a therapeutically effective or prophylactically effective amount of said purified compound to a mammal which has or is at risk for developing a condition which is benefited by the inhibition of the activity of a proinflammatory factor.

13. A method according to claim 12, wherein said condition is a cyclooxygenase-2 mediated condition.

14. A method according to claim 13, wherein said cyclooxygenase-2 mediated condition is general inflammation.

15. A method according to claim 13, wherein said cyclooxygenase-2 mediated condition is arthritis.

16. A method according to claim 13, wherein said cyclooxygenase-2 mediated condition is pain.

17. A method according to claim 13, wherein said cyclooxygenase-2 mediated condition is cancer.

18. A method according to claim 12, wherein said purified compound is administered as a pharmaceutical composition comprising a pharmaceutically acceptable excipient.

19. A method according to claim 12, wherein said purified compound is administered as a nutritional composition comprising a nutritionally acceptable excipient.

20. A method according to claim 12, wherein said purified compound is administered as a food composition.

21. A method according to claim 12, wherein said purified compound is administered as a food ingredient composition.
